(19) United States
(12) Patent Application Publication
(10) Pub. No.: US 2002/0004033 A1
(43) Pub. Date: Jan. 10, 2002

(54) EMULSION TYPE VAPOR GENERATOR

(76) Inventor: DIRK SORGENFREY, CALDWELL, NJ (US)

Correspondence Address: STEPHAN A PENDORF, PENDORF & CUTLIFF, PO BOX 20445, TAMPA, FL, 336220445

(21) Appl. No.: 09/296,220

(22) Filed: Apr. 22, 1999

(51) Int. Cl. C09K 3/00; B01F 3/08; B01F 17/00; A61L 9/02; A61K 9/72; A01N 25/06; A01N 25/18; A01N 25/20; A61K 9/00; A01N 25/02; C07C 69/52

(52) U.S. Cl. 424/43; 424/40; 424/45; 424/400; 560/205; 516/21; 516/22

(57) ABSTRACT

An emulsion type composition formulated to provide consistent, balanced and sustained release of vapors. In comparison to prior art gel type compositions, the composition does not solidify and remains a thick liquid at room temperature. The emulsion type vapor generator is characterized by shrinking one-dimensionally. The composition can be formulated to release vapors of active ingredients such as air-fresheners, medicaments, decongestants, inhalants, repellants, insecticides, bactericides, deodorants, and the like, at a uniform rate and for a sustained period of time.

### FIELD OF THE INVENTION

The present invention concerns an emulsion type composition formulated to provide a balanced, sustained release of vapors. The composition can be formulated to release vapors of active ingredients such as air-freshener aromatics, medicaments, decongestants, inhalants, repellants, insecticides, bactericides, deodorants, and the like, at a uniform rate and for a sustained period of time.

### BACKGROUND OF THE INVENTION

In the early days of air treatment, a “hand gun” was used to atomize a suitable liquid. This gave way to pressurized containers that used propellants such as chlorofluorocarbons or the like to propel the liquid into the air in the form of an aerosol.

This “active” propellant technique has become less popular due to the increasing public awareness of the hazardous effects of the propellants on the environment and human beings. Further, there are retail disadvantages involved with storing a large inventory of pressurized containers, with the ever-present danger of explosion.

A technique that is gaining favor is known as the “static” technique. This technique does not use a pressurized propellant. Rather, this static technique depends upon the volatility characteristic of many materials at ambient temperature and atmospheric pressure. An intermittent motor-driven fan can be used to improve the dissemination of the vapors.

Several classes of static vapor generators are in use. The “wick” type vapor generator uses a bottle that is filled with the liquid to be vaporized, the bottle also contains a wick material whose capillary characteristic causes the liquid to rise up into the wick to a level at which the liquid may be evaporated into the air. In this way a suitable vapor is produced. The wick method has a number of disadvantages. For example, the evaporation slows down as the liquid level in the bottle drops, because capillary action is offset by gravity as column height increases, thus resulting in progressively decreasing effectiveness. Another problem arises from the fact that liquids to be vaporized are generally mixtures of more volatile and less volatile molecules, and different air treatment liquids tend to vaporize (evaporate) at different rates. In the case of fragrances, for example, less volatile components (“bottom notes”) of the fragrance composition tend to accumulate on the wick over time. This progressively reduces the effective surface area for the more volatile components (“top notes”) to evaporate from and consequently shifts the balance of the fragrance notes emitted into the atmosphere towards weaker bottom notes. This can result in undesired changes in the perceived fragrance “bouquet” over time (FIG. 1a and 1b).

Other air treatment properties may be effected similarly. Many vapor generators are formulated with two or more classes of active substances, such as a combination of air freshening ingredient and air sanitation ingredient, with the intention, that the expiration of the air freshening ingredient signal provides an olfactory indication of the expiration of release of the sanitation ingredient. Where the two classes of materials have different vaporization rates, the expiration of, e.g., a fragrance material cannot be used as a reliable indicator of the expiration of, e.g., a disinfecting material.

In an attempt to overcome the disadvantages of the wick/bottle arrangement, resort was made to a porous so-called “absorption block” that could be formed of wood, stone, ceramic, fiber, plastic, etc. By immersing such an absorption block into a suitable air treatment liquid or mixture, it would absorb a given volume thereof, and since the absorption block has a large “wicking area”, the above discussed accumulation of low volatile components is minimized. However, the absorption block still has the inherent problem of different vaporizing rates, and, moreover, its absorption volume is limited by the size of the absorption block.

One approach to overcoming the problems of the wick type vapor generator, as well as those of the absorption block type, was to use the “drip” technique, wherein a bottle of liquid is inverted to permit the contained mixture of liquids to drip into a suitable recess for vaporization. This drip technique introduced the danger of leakage and spillage.

Yet another class of vapor generator is known as the “gel” type. Gel type vapor generators comprise of three main ingredients: (1) a liquid such as water, (2) gelling agents such as CAB-O-SIL, Xantham, alginate, agar agar, gelatin, gum tragacanth, etc., and (3) the active air treatment ingredient to be vaporized (fragrances, medicaments, decongestants, inhalants, repellants, insecticides, bactericides, deodorants, and the like). Gels are typically prepared by a special procedure necessitated by the gelling characteristics of the gelling agent. Most procedures require heat at some point, but certain gelling agents, such as KELZAN (Monsanto industrial xantham gum), allow gel preparation without heat. Typically the gel will include, by weight, on the order of about 0.3% to 3.0% gelling agent and about 80% to 90% water, with active liquid ingredients as the balance. As the gel solidifies it assumes the form of the receptacle into which it has been poured.

There is an interesting phenomenon associated with some of the early gel type vapor generators. When such a gel body is exposed to the air, the exposed exterior surface permits the liquids to vaporize, and in this way the desired vapors are generated for dissemination throughout the space to be treated. The increasing weight loss causes the gel to lose volume—it shrinks or contracts. The shrinkage can be likened to the squeezing of a sponge: the contraction squeezes liquid out of the gel—which tends to be the fragrance oils that have accumulated near the surface of the gel, since water volatilizes more readily than most fragrance oils. This bleeding-out effect is commonly referred to as “syneresis”.

Syneresis is an unpleasant sight and most air fresheners are formulated to avoid it. In modern “jelly jar” type vapor generators the syneresis problem is circumvented by choosing a suitable gelling agent and by keeping the amount of active ingredient small enough. This, however, tends to result in reduced potency.

For example, U.S. Pat. No. 3,969,280 (Sayce, et al.) entitled “Solid air freshener gels” relates to a solid air freshener gel composition utilizing a cross-linked carboxymethyl cellulose (CMC) as the primary gelling agent. Sayce, et al. are concerned with the problem of syneresis, and teach an air freshener gel which is substantially syneresis free. It comprises, in an aqueous system, from 0.5-10% by weight of CMC, 0.01-10% by weight of perfume, and a source of trivalent cations selected from chromic ion, aluminum ion or mixtures thereof.

It had also been proposed that the syneresis problem in carrageenan based solid air freshener compositions can be alleviated by using sufficiently high percentages of the carrageenan gelling agent. However, its high cost renders it very difficult to produce an economic solid air freshener gel without having significant amounts of syneresis.

In U.S. Pat. No. 4,166,087 (Cline, et al.) the inventors teach the use of a gel for air treatment, said gel being provided in a can. Vaporization first takes place through only the exposed upper surface of the gel, allowing limited vaporization. As the gel progressively dries, the gel body shrinks not only along its vertical axis (downward) but also in diameter, pulling away inwardly from the sidewall of the can (see, for example, FIGS. 2A-2C of Cline et al.). This three-dimensional shrinkage results in the formation of an annular air space between the gel body and the can inside wall, enlarging the evaporative surface of the gel body, thus increasing the rate of vaporization. Thereafter, as the gel body continues to shrink, the above-discussed syneresis action progressively squeezes the fragrance oils out of the gel, and non-evaporated oils tend to gravitate downwardly into a blotter-like or felt-like absorbent “syneresis pad”.

In theory, the constant decrease in evaporative surface area of the gel body of Cline et al. is offset by the increase in evaporative surface area from the syneresis pad. This is supposed to provide a constant vapor output concentration of the vapor generator (sometimes referred to as “vapor density”), until most of the gel body has disappeared. However, this simplified formula only holds true for a certain small range of ratios between the height and diameter of the can. Moreover, the syneresis occludes the surface of the base of the gel, which impedes the vaporization of water. Therefore, the base diameter does not shrink as much as the top diameter of the gel body. This is also evident from FIG. 2C of Cline et al., which quite accurately depicts the final stages of a gel type vapor generator: The syneresis pad is largely covered by the increasingly cone-shaped remains of the gel body. Therefore, the syneresis pad cannot sufficiently compensate for the loss in surface area from the gel body.

Another disadvantage of gel type vapor generators is the fact that they tend to harden as they contract, especially at their surfaces, which eventually will trap some air treating material within the gel body.

More recently U.S. Pat. No. 5,498,436 (Modliszewski, et al.) entitled “Coprocessed galactomannan-glucomannan” issued teaching a composition comprising: (A) a coprecipitate consisting essentially of: (i) a galactomannan, with (ii) a glucomannan; and (B) optionally, a gelling agent admixed with the formed coprecipitate. The inventive compositions are purportedly useful primarily as the base for many food and industrial products. There is mention of suitability for use in an air freshener gel formulation; however, no formulations or examples of air fresheners are included in the specification.

It appears that development of gel type vapor generators has reached a dead end. Modern gel type vapor generators, known as “jelly jar” type vapor generators, are meant to be free standing units and, therefore, try to avoid syneresis, because of its unpleasant sight, potential spillage, and/or even toxicological concerns. Further, for purely esthetic reasons, the choice of thickeners as well as fragrance oils is limited to those that result in a clear product. The jelly jar vapor generators are esthetically more appealing, but they do not perform as well as their predecessors, because gel type vapor generators, which allow syneresis to occur, expose the oils directly to the surrounding air.

There is thus a need for a new class of vapor generator that can provide a balanced release of vapor components at a uniform rate and for a sustained period of time. There is also a need for a new class of vapor generator, which can provide a balanced release of two or more different types of active ingredients such as air-fresheners, medicaments, decongestants, inhalants, repellants, insecticides, bactericides, deodorants, and the like, over a wide range of situations including, for example, food markets, restaurants, kitchens, restrooms, dressing rooms, laboratories, retail stores, offices, waiting rooms, laundries, theaters, etc. Moreover, the vapor generator should operate effectively for an acceptable time period without requiring attention or maintenance.

### SUMMARY OF THE INVENTION

The present invention solves the above and other problems by providing a new class of vapor generator which, because of its consistency, will be referred to hereafter as an emulsion type vapor generator.

The new class of vapor generator is characterized by three main components:

(A) water as main ingredient in terms of weight;

(B) an active air treatment ingredient to be vaporized, for example, fragrances, medicaments, decongestants, inhalants, repellants, insecticides, bactericides, deodorants, and the like, which is emulsified into the water; and

(C) an emulsifying agent, which may also be referred to as a viscosifier or emulsifier, as known in the art of forming emulsions, in sufficient amount to keep component (B) emulsified in component (A) and in sufficient amount to form a emulsion consistency, without formation of a solid. The emulsifying agent is preferably a polymer formed from monomers selected from ethenoid monomers containing acrylic or methacrylic groups such as dimethylaminoethyl methacrylate (DMAEMA), dimethylaminopropyl acrylamide, diethyl-aminoethyl methacrylate and dimethylaminoethyl acrylate, or other ionogenic methacrylate esters containing vinyl and tertiary amine grouping, with DMAEMA being preferred.

The components are formulated into a composition classified in analogous arts, such as the cosmetic or food preparation art, as an emulsion. Thermodynamically unstable in nature, an emulsion is a system comprised of two immiscible liquid phases, one of which is dispersed in the other with the help of an emulsifying agent. The droplet size of the dispersed system can range from 0.01 μm to 100 μm, or more typically from 0.1 μm to 10 μm. The term “emulsion” is used in the context of the present invention to refer to a composition, which a) maintains one phase dispersed within the other phase during its shelf life—not necessarily as droplets -, and b), will shrink one-dimensionally, i.e. gradually settles downwards as the emulsion loses volume, after having been poured or pumped into a can and exposed to the atmosphere. This one-dimensional shrinkage occurs predominantly at the surface only. The composition will not form a solid in the manner of a gel. A gel, when provided in a can, will shrink three-dimensionally and thus will (1) shrink downwards along its entire vertical axis and (2) shrink in diameter, and thus will pull away from the sidewall of the can and expose different areas of surface area over time, from initially exposing only the upper surface, to exposing the upper surface plus sidewalls, followed by a gradually shrinking surface area as the gel block dries.

Surprisingly, the present invention provides a more uniform, sustained, and balanced release of vapors than possible with either the gel type vapor generators or the wick type vapor generators by taking advantage of the novel release characteristics.

That is, using can-style vapor generators for purposes of comparison, upon removal of the lid the emulsion type vapor generator will initially expose the top surface of the emulsion to the atmosphere. Since water volatilizes more readily than most fragrance oils, fragrance oils will accumulate near the surface of the gel. Over time the accumulating fragrance oils will suppress the vaporization of water (occluding effect) until equilibrium is reached. Since the emulsion does not shrink in diameter, but predominantly along its vertical axis, the fragrance release is uniform from the time the equilibrium is reached until the emulsion is spent, because the vapor generating surface area remains the same during that time period. As the surface layer of the composition gradually moves downward, the layer continues to expose new high volatility ingredients as well as low volatility ingredients. Thus, the uniformity and intensity of the fragrance notes released by this formulation remains relatively constant.

In contrast, in the prior art gel type syneresis vapor generators as described in U.S. Pat. No. 4,166,087 (Cline, et al.) vaporization first takes place through only the exposed upper surface of the gel, providing a limited vaporization. Then, as the gel progressively dries, the gel body shrinks three dimensionally, pulling away inwardly from the sidewall of the can, forming an annular air space between the gel body and the can inside wall. In view of the life time of a gel type vapor generator, the separation from the sidewall is somewhat sudden, resulting in a sudden significant increase in the evaporative surface area of the gel body, and consequently, increasing the rate of vaporization. Thereafter, as the gel body continues to shrink, the above-discussed syneresis action progressively squeezes the fragrance oils out of the gel, and non-evaporated oils tend to gravitate downwardly into a blotter-like or felt-like absorbent “syneresis pad”. Once the gel is detached from the sidewall, the evaporative surface area of the gel decreases, which is not fully compensated for by an increase in surface area of the syneresis pad as suggested by Cline et al. (see discussion above). In addition, the gel type vapor generator tends to harden as it shrinks, thus trapping fragrance in its matrix.

The present invention, in contrast, provides a vapor generator which, in comparison to the gel type vapor generator, provides a more uniform release of vapors, and in addition provides a balanced release of vapors. Moreover, since the emulsion type does not harden, fragrance material is not likely to get trapped, which increases the yield.

The emulsion type vapor generator also provides greater precision when a fragrance component is used as an indicator of a non-fragrant active substance such as an insecticide. As can be seen from the above discussion, the last release of aromatic will coincide with the last release of insecticide, and once fragrance is lost, the generator can be considered spent. The present invention thus represents an improvement over the above-described gel type generator in this regard also.

Further, the novel emulsion type vapor generator provides an inexpensive fragrance delivery solution to the industry. It is to be noted that the emulsifying agent need comprise only a small percentage of the total emulsion composition; the main ingredients being first water, and, usually second, fragrance.

Finally, the product is easy to handle, is spill proof, and flash point is not a safety issue unlike for some wick type vapor generator compositions. The product is therefore safe for shipping and handling, i.e. which is especially critical, when the air freshening containers are being replaced.

In comparison to the modern jelly jar type vapor generators, the emulsion type vapor generator can be loaded with a higher concentration of active agent. That is, the jelly jar type vapor generator avoids the syneresis problem by using less fragrance oils than would produce syneresis.

The product can be formulated to provide anywhere from, preferably, one month to three months of vapor release, and to provide an even and balanced release of air treatment agent. It can thus easily outlast both the wick type as well as the gel type vapor generator in terms of intensity and balance in fragrance. Obviously, longer and shorter vapor release periods can be easily provided by simply modifying (1) the evaporative surface area and (2) the total volume of emulsion (length of vertical axis).

The foregoing has outlined rather broadly the more pertinent and important features of the present invention in order that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the specific embodiments disclosed may be readily utilized as a basis for modifying or formulating other vapor generator or release systems for carrying out the same purposes of the present invention. It should also be realized by those skilled in the art that such equivalent formulations do not depart from the spirit and scope of the invention as set forth in the appended claims.

### BRIEF DESCRIPTION OF THE DRAWINGS

For a fuller understanding of the nature and objects of the present invention reference should be made by the following detailed description taken in with the accompanying drawings in which:

FIG. 1a shows the change in concentration of a three component mixture. D-limonene (39.5%) is a highly volatile compound, dihydromyrcenol (32.5%) is a somewhat volatile compound, and isopropyl myristate (IPM; 28.0%) is an almost non-volatile compound. As can be seen, D-limonene is quickly replaced on the wick by the less volatile compounds. Equilibrium will eventually be reached with IPM constituting over 90% of the composition at the wick surface in this particular case.

FIG. 1b shows the weight loss of the three component mixture over time, which mirrors the events depicted in FIG. 1a.

FIG. 2 shows a weight loss pattern (gram/day) of an emulsion type vapor generator.

FIG. 3 shows a weight loss pattern of an emulsion type vapor generator versus a jelly jar type vapor generator.

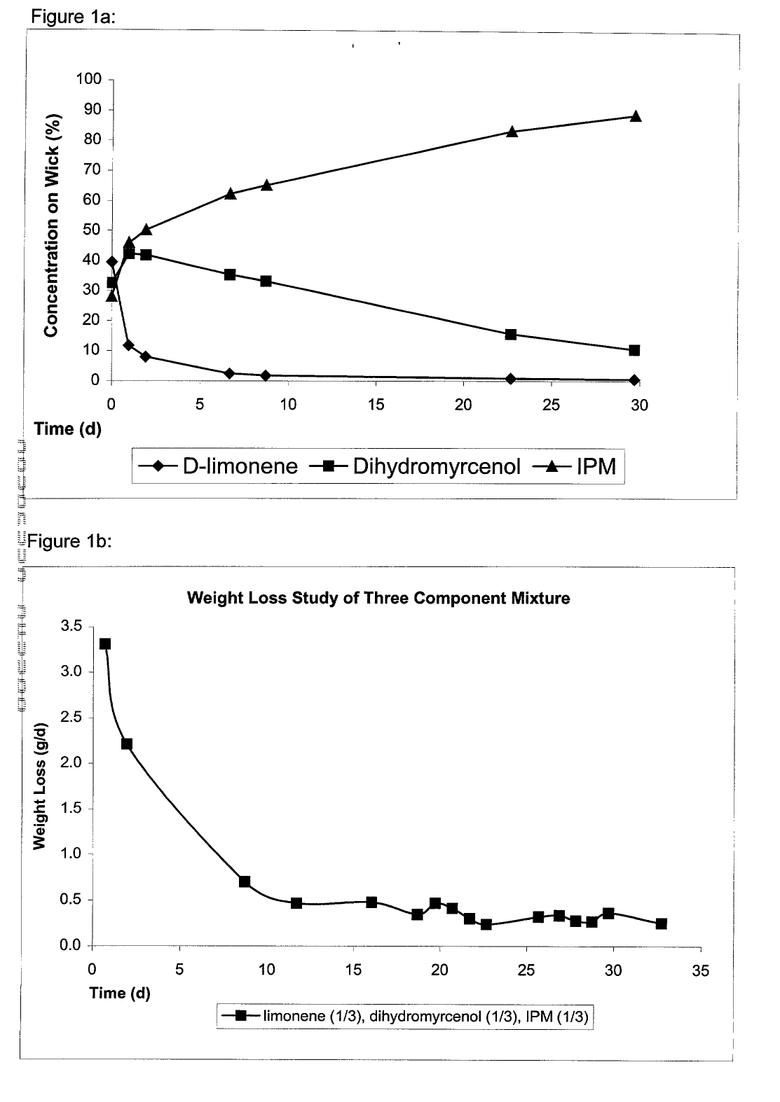

### DETAILED DESCRIPTION OF THE INVENTION

The present invention provides an “emulsion” type vapor generator. As used herein, an emulsion type vapor generator means a composition which has three essential ingredients, namely, (A) water as main ingredient in terms of weight; (B) at least one hydrophobic air treatment ingredient to be vaporized, for example, fragrances, medicaments, decongestants, inhalants, repellants, insecticides, bactericides, deodorants, and the like, this hydrophobic component emulsified into the water; and (C) an emulsifying agent, which may also be referred to as a viscosifier or emulsifier, as known in the art of forming emulsion, in sufficient amount to keep component (B) emulsified in component (A) and sufficient amount for formation of an emulsion consistency, without, however, forming a solid. In use, the emulsion type vapor generator is preferably provided in a solid receptacle, such as a can or jar, with a removable lid. Removal of the lid exposes the upper surface of the emulsion to the atmosphere.

The emulsifying agents (surfactants, viscosifiers, thickening agents, emulsifiers) can be any single agent or any mixture of agents, as known in the cosmetic, food preparation or pharmaceutical arts, which results in an oil in water emulsion, i.e. which at least temporarily (months; during storage) maintains the oil phase dispersed in the water phase and prevents the two phases from separation. It is implicit that the emulsifying agents stabilize the oil droplets of the oil phase in the water phase; the emulsifying agents do not dissolve the oil in the water to form a solution.

Exemplary emulsifying agents include a polymer (homopolymer or copolymer) formed from monomers, at least one of which is selected from ethenoid monomers containing acrylic or methacrylic groups such as dimethylaminoethyl methacrylate (DMAEMA), dimethylaminopropyl acrylamide, diethyl-aminoethyl methacrylate and dimethylaminoethyl acrylate, or other ionogenic methacrylate esters containing vinyl and tertiary amine grouping, with DMAEMA being preferred. However, other emulsifying agents well known in the cosmetic, drilling, and food preparation arts may be used if they result in an oil in water emulsion.

Other components may be included so long as they do not detract from the intended manner of operation of the emulsion type vapor generator. For instance, if the non-evaporative portion of the oil phase, including the emulsifying agents, is too high, the yield of the vapor release could be reduced. This also would render the end-point detection more difficult.

The emulsion type vapor generator composition is a composition which, when poured into a receptacle and exposed to the atmosphere, will shrink predominantly one-dimensionally, that is, will gradually settle downwards along its vertical axis as the emulsion loses volume. The shrinkage occurs predominantly at and immediately below the surface of the emulsion with the remainder of the emulsion staying unchanged. The composition will not form a solid in the manner of a gel. A gel, in comparison, is a composition which when provided in a receptacle, will solidify and shrink three-dimensionally and thus will (1) shrink downwards along its entire vertical axis and, in addition, (2) shrink in diameter. It therefore will exhibit changes in vaporization surface area from initially exposing only the upper surface, to exposing a larger surface area to the atmosphere as it pulls away from the sidewall of the receptacle, to finally exposing an ever smaller vaporization surface area as the gel contracts.

Using a fragrance vapor generating composition as an example with which to illustrate the present invention, the emulsion is formulated to comprise 1-30 wt % fragrance oil; 0.1-10 wt % polymer (e.g., Salcare SC91); and water to make 100 wt %. The product is preferably white in appearance. This composition is not only economical in terms of ingredient costs, it is also easy to make. The sample is prepared by mixing the emulsifying agent, the fragrance oil and the water without heating, which may be at room temperature. No specific equipment or conditions are required for stirring—mere shaking of a bottle by hand or stirring sufficiently to keep ingredients in motion is sufficient. The viscosity may initially be as low as that of water for about 15 to 20 minutes, after which it will increase gradually. After about 30 to 40 minutes one can hold a jar of the emulsion upside down without product spillage. However, the product exhibits sensitivity to shear. This is a benefit—the product is stiff when it sits, but it behaves like a liquid when stirred or pumped.

The finished product is a stabilized oil-in-water emulsion. The emulsifying agent has the crucial stabilizing role of keeping the fragrance dispersed in water during storage and thus preventing formation of separate oil and water layers.

In comparison to gel formulations, which had to be mixed, heated, then rapidly molded prior to solidification, the emulsion of the above example is easy to mix, does not solidify into a gel (though there is often a noticeable increase in viscosity during and after mixing), and can easily be re-formulated if necessary. Emulsifying agents requiring heat during the manufacturing process can be used, but in that case this particular benefit over gels does, of course, not exist.

During use (i.e., upon exposure to air), water and fragrance gradually evaporate from the top surface of the emulsion. Since water volatilizes more readily than most fragrance oils, fragrance oils will accumulate near the surface of the gel. The emulsion may break at the surface depending on the oil load with respect to the emulsifying agent. Over time the accumulating fragrance oils will suppress the vaporization of water (occluding effect) until equilibrium is reached. Since the emulsion does not shrink in diameter, but predominantly along its vertical axis, the fragrance release is uniform from the time the equilibrium is reached until the emulsion is spent, because the vapor generating surface area remains essentially the same during that time period. More importantly, as the surface layer of the composition gradually moves downward, the layer continues to expose new high volatility ingredients as well as low volatility ingredients. Thus, the uniformity and intensity of the fragrance notes released by this formulation remains relatively constant.

The emulsion type vapor generating composition remains balanced in fragrance notes much better than a wick system, because the less volatile oils do not interfere as they do in a wick system. That is, in wick type vapor generators a balanced mixture of high and low volatile oils initially travels up the fresh wick. Over time, low volatile components of the mixture (bottom notes) tend to accumulate at the wick surface, thus effectively reducing the surface area to which the balanced mixture can travel up to. This eventually results in the emanation of a weak, bottom note rich fragrance. Likewise, gel type vapor generators not only tend to be weak to begin with, but they also emanate less and less of the top notes over time. Even if syneresis occurs, eventually it ceases—before the gel is spent—and remaining top notes are trapped in the gel matrix as the gel gets harder and harder with continuous contraction.

It is possible to provide a pad at the bottom of the container of the emulsion type vapor generator, but this is provided mainly for preventing any spillage of any residual oils towards the end of its life cycle during use. Since the emulsion type composition will shrink one dimensionally, oils will not accumulate on the pad until all of the emulsion is spent. Therefore, the pad is not used to increase the vaporization rate as intended by Cline, et al.

The emulsion type vapor generating composition can be loaded with more fragrance oil than the modern jelly jar vapor generator, and thus can achieve the same or better effect from a smaller container.

The emulsifying agent mentioned in the above example, which can be used in the present invention, can be any of those used to produce a stable oil-in-water emulsion in the paint industry, the skin care industry, and the food preparation industry, and related arts. However, the use of such an emulsifying agent to form an emulsion type vapor generating composition is new.

When used in industrial air freshening units, the receptacle (e.g., a can) with the product will be sitting in a conventional air freshening generator, typically a wall unit that comes with a battery driven (intermittent) ventilation system. It has been noted that, after the product has been spent, there may be some residual oil phase (low volatile oils and/or waxes, etc.) and emulsifying agents. However, this is not considered to be a problem, since an absorbing pad can be employed.

Thus, for industrial air freshening units, the product of the present invention is stable and requires little or no attention other than replacement at predetermined intervals. The product typically loses more of its weight than gel type vapor generators containing the same oil load, thus increasing the yield of fragrance release.

The present invention can also be made available for home use. It would be preferred to use a composition with an oil load, which would avoid water phase and oil phase separation at the surface in order to minimize the danger of spillage.

The three main components of the present invention will now be discussed in greater detail.

(A) Liquid Component

The first component is a liquid, which does not interfere with the active ingredient(s) and evaporates to cause the active agent(s) to be expelled from the emulsion type vapor generator composition. For cost and environmental considerations, water is preferred as the most economical and ecological liquid.

(B) Active Air Treatment Ingredient

The second component is the active air treatment ingredient, and may be any of the conventional ingredients used in air-fresheners, medicaments, decongestants, inhalants, repellants, insecticides, bactericides, deodorants, and the like.

A variety of fragrance oils may be incorporated into the stable fragrance delivery system according to the present invention. These fragrance oils include natural and synthetic aromatic compounds and/or oils, oleoresins and extracts derived from plants, leaves, flowers, fruits and so forth, and combinations thereof. These fragrance oils may be used individually or in a mixture as is well known in the art.

The chemicals or substances which are used to produce a desired fragrance may be any one or more of those which are commonly used by those skilled in the art of fragrance chemistry or perfumery, some of which are listed in the following texts:

Robert R. Calkin, J. Stephan Jellinek, Perfumery, Practice & Principle, John Wiley and Sons, Inc. New York, 1994;

Rüdiger Hall, Dieter Klemme, Jürgen Nienhaus, Guide to Fragrance Ingredients, H&R Edition, R. Gross & Co. Publishing, Hamburg, 1985;

Julia Müller, The H&R Book of Perfume, H&R Edition, Johnson Publications, Ltd., London, 1984;

Fragrance Guide-Feminine Notes, Masculine Notes, H&R Edition, R. Gross & Co. Publishing, Hamburg, 1985;

each of which is incorporated herein by reference.

It is specifically intended that the present invention not be limited to any particular fragrance or combination of fragrances, whether known or discovered in the future since any fragrance or chemical substances which humans find pleasant and desirable to inhale are within the scope of the present invention.

Specific examples of fragrance materials suitable for use in the present invention, and which generally comprise a range of active molecules of varying vapor pressures from very low to very high, include:

TABLE 1
Natural Fragrances

Ambrette seed
Armoise
Basil
Bay
Benzoin Siam
Bergamot
Birch tar
Camomile Roman
Cardamon
Cassis bourgeons (base)
Castoreum
Cedarwood Virginian
Celery seed
Cinnamon leaf
Cinnamon bark
Cistus oil
Civet
Clary sage
Clove bud
Coriander
Costus (base)
Cumin
Estragon
Galbanum oil
Geranium Bourbon
Guaiacwood
Iris concrete
Jasmin absolute
Labdanum extract
Lavender
Lavandin
Lemon
Lemongrass
Lime West Indian
Mandarin
Mimosa absolute
Neroli
Nutmeg
Oakmoss absolute
Olibanum extract
Opoponax extract
Orange sweet
Patchouli
Pepper
Peppermint
Peru balsam oil
Petitgrain Paraguay
Pimento
Rose oil
Rose absolute
Rosemary
Rosewood
Sandalwood East Indian
Styrax oil
Tagete
Tonka absolute
Tuberose absolute
Thyme
Vanilla absolute
Vetyver Bourbon
Violet leaf absolute
Ylang extra

TABLE 2
Synthetic Fragrances

Acetophenone
Aldehyde C₁₀
Aldehyde C₁₁ undecylenic
Aldehyde C₁₂ lauric
Aldehyde C₁₂ MNA
Aldehyde C₁₄ (gamma-
undecalactone)
Aldehyde C₁₆
Aldehyde C₁₈ (gamma-
nonalactone)
Allyl cyclohexyl propionate
Ambroxan
Amyl cinnamic aldehyde
Amyl salicylate
Anisaldehyde
Aurantiol
Benzaldehyde
Benzyl acetate
Benzyl salicylate
Brahmanol
Calone
Cashmeran
Cedramber
Cedryl acetate
Cinnamic alcohol
Citral
Citronellal
Citronellol
Citronellyl acetate
Coumarin
Cyclamen aldehyde
Cyclopentadecanolide
Damascone beta
Dihydromyrcenol
Dimethyl benzyl carbinyl
acetate
Diphenyl oxide
Ethyl phenylacetate
Ethyl vanillin
Eugenol
Evernyl
Frambinone
Galaxolide
gamma-Decalactone
Geraniol
Geranyl acetate
Geranyl formate
Geranyl nitrile
Greenyl acetate
Hedione
Helional
Heliotropin
cis-3-Hexenyl acetate
cis-3-Hexenyl salicylate
Hexyl cinnamic aldehyde
Hexyl salicylate
Hivertal
Hydroxycitronellal
Indol
Ionone alpha
Isobornyl acetate
Iso butyl quinoline
Isoeugenol
Iso E super
Isogalbanate
cis-Jasmone
Lilial
Linalool
Linalyl acetate
Lyral
Maltol
Methyl Anthranilate
Methyl benzoate
Methyl cinnamate
Methyl chavicol
Methyl ionone gamma
Methyl napthyl ketone
Methyl octine carbonate
Methyl salicylate
Musk ketone
Musk T
Paracresyl acetate
Phenoxyethyl isobutyrate
Phenylacetaldehyde
Phenylacetic acid
Phenylacetaldehyde dimethyl
acetal
Phenylethyl acetate
Phenylethyl alcohol
Phenylethyl dimethyl carbinol
Phenylethyl phenylacetate
Phenylpropyl alcohol
Rosalva
Rosatol
Rose oxide
Sandela
Styrallyl acetate
Terpineol
Tonalid
Vanillin
Vertacetal
Vertofix
Vetiveryl acetate
Vertenex (PTBCHA)

It is also intended that for certain applications, chemicals may be incorporated into the formulation alone or in combination with fragrance producing chemicals, even though they themselves do not produce a detectable scent but instead produce certain biological or psychological effects. These substances include, but are not limited to medicaments, decongestants, inhalants, repellants, insecticides, bactericides, deodorants, pheromones which may alter the physiology of the body, mood-altering substances, organ extracts, plant extracts, or other materials or chemicals which provide a desired biological or psychological effect.

It will be recognized that fragrance chemicals may be mixed or combined with various solvents, diluents, oils, waxes, surfactants, or other substances which act to dissolve the fragrance chemicals or alter their intensity, stability, viscosity, rate of release or other physical or chemical characteristics. However, to reduce air pollution (particularly when the vapor generator is used in a closed indoor space) and to maximize the environmental friendliness of the present vapor generating composition, non-aromatic volatile organic chemicals and such are preferably excluded from the emulsion type vapor generator.

(C) Emulsifying Agent (Viscosifier, Emulgator, Emulsifier, Suspension Agent) Component

The third essential component of the present invention is the emulsifying agent. This can be any agent used for this purpose in the cosmetic, pharmaceutical or food preparation arts, and preferably resulting in an emulsion possessing the following characteristics:

(1) it shrinks generally one-dimensionally (that is, shrinks downward along its vertical axis, ignoring any cracks or fissures which may form with low oil loads), after been poured or pumped into a receptacle and exposed to the atmosphere;

(2) it does not form a three dimensional matrix or coherent body, which can be picked up and put down without noticeable change in shape and form like a gel type vapor generator;

(3) it cannot be parted into two bodies along a shear plane like a gel type vapor generator;

(4) it can be stirred and pumped at room temperature even as a finished product, ready for use

(5) the emulsion can be provided to commercial or domestic consumers in convenient refill pouches, the contents of which can be poured or squeezed out into a reusable receptacle; this represents an improvement over gel type vapor generators which can not be refilled by the consumer, and

(6) the emulsion is more completely evaporated and leaves less residue as compared to a gel type vapor generator.

The exemplary emulsifying agent for the emulsions of the present invention is preferably employed at levels ranging from about 0.1 to about 10% by weight of the total emulsion, and most preferably from between about 0.5 to about 4.0% by weight of the emulsion. There is no particular limitation on the amount of emulsifying agent employed, so long as it is adjusted to provide the desired emulsion properties sought for the purposes of the present invention. The preferred emulsifying agent of the present invention is a dimethylaminoethyl acrylate (DMAEMA) homopolymer or copolymer. DMAEMA is hydrophilic ethylenically unsaturated monomer, an ionogenic methacrylate ester containing a vinyl and a tertiary amine grouping. It is a relatively non-volatile, clear liquid which is very soluble in water and in common organic solvents. Other ethenoid monomers containing acrylic or methacrylic groups such as dimethylaminopropyl acrylamide, diethylaminoethyl methacrylate and dimethylaminoethyl acrylate are also contemplated by the present invention with DMAEMA being the more highly preferred.

DMAEMA is well known. U.S. Pat. No. 4,281,175 (Kametani, et al.) entitled “Process for producing dimethylaminoethyl methacrylate” teaches transesterification of methyl methacrylate with dimethylaminoethanol using alkyltin compounds as catalysts. Typical alkyltin compounds used are tetrabutyltin, trioctyltin ethoxide, dibutyltin dimethoxide, dibutyltin dihydride, dibutyltin dilaurate, dibutyltin maleate, bis(tributyltin) oxide and bis(dibutylmethoxytin) oxide. JP52153912 (Kametani, et al.) entitled “Process for producing dimethylaminoethyl methacrylate” teaches preparation of DMAEMA in high yield by the reaction of methylmethacrylate with dimethylaminoethanol in the presence of cyclopentane and its metal complex as a polymerization inhibitor.

JP52153911 (Kametani, et al.) entitled “Process for producing dimethylaminoethyl methacrylate” teaches preparation of DMAEMA by the ester interchange of methylmethacrylate with dimethylaminoethanol in the presence of specific catalysts which are not inactivated during the reaction.

JP52153910 (Kametani, et al.) entitled “Process for producing dimethylaminoethyl methacrylate” teaches preparation of DMAEMA by the ester interchange reaction of methylmethacrylate with dimethylaminoethanol in the presence of specific catalysts.

U.S. Pat. No. 4,052,343 (Cunningham) entitled “Poly(dimethyl-aminoethyl methacrylate) and method of preparation” teaches crosslinked ion exchange resins derived from dimethylaminoethyl methacrylate.

U.S. Pat. No. 5,717,045 (Tseng) entitled “Crosslinked copolymers of vinyl pyrrolidone and dimethylaminoethyl methacrylate and process for making same in aqueous solution having desirable gel properties” teaches crosslinked copolymers of vinyl pyrrolidone (VP) and dimethylaminoethyl methacrylate (DMAEMA) for use in personal care products, made in aqueous solution by copolymerization of VP and DMAEMA monomers in aqueous solution using 1-vinyl-3-(E)-ethylidene pyrrolidone (EVP) as the crosslinking agent. The product is a one-phase, aqueous gel of the copolymer, which is useful in hair care products.

German Patent DE 4125048 (Albrecht) entitled “Retard pharmaceutical prepn. for periodontitis treatment—contains anti-periodontitis drug esp. metronidazole, in cationic (meth)acrylic] polymer matrix pref, used as film strip” teaches drug release-retarded pharmaceutical preparations containing 10 wt.% of an anti-periodontitic drug in a cationic (meth)acrylic polymer (copolymerisate of dimethylaminoethyl methacrylate and neutral methacrylate). A drug delivery “patch” can be left in place for two days.

The teachings of each of the above patents are incorporated herein by reference.

A preferred commercially available formulation of emulsifying agent which can be directly employed in the present invention is marketed under the trademark SALCARE, (pseudo mark Salon Care) by Allied Colloids Limited of Bradford West Yorkshire, United Kingdom. A range of SALCARE emulsifying agents are available, including SALCARE SC10, SC91, SC92, SC95, and SC96. Preferred for use in the present invention is SALCARE SC91. SALCARE SC91 is a sodium polyacrylate dispersed in a highly refined cosmetic grade mineral oil. SALCARE SC95 is a high cationic charge DMAEMA homopolymer dispersed in mineral oil.

(D) Additional ingredients

Any additional ingredients not inconsistent with the desired emulsion consistency can be included in the emulsion formulation. Examples of such additional ingredients include surfactants, oils, waxes, and additional thickeners. However, it is preferred, that additives be excluded or minimized, which do not contribute to or protect the desired emulsion properties sought for the purpose of the present invention.

Battery powered ventilation systems wherein a fan is activated intermittently are well known and need not be described here.

The emulsion can also be provided in pouches for refilling the reusable receptacle. The emulsion leaves little residue (usually 20% or less, preferably 10% or less), which can be easily cleaned out prior to refilling with fresh emulsion from the refill pouch. This reuse of the receptacle represents an ecological advantage of the present invention over the gel type vapor generators, which can not be refilled with gel, since the gel is a solid, not a liquid.

The present invention will now be described by reference to an air freshener.

### EXAMPLE 1

2.0% SALCARE SC91 and 20.0% fragrance oil (the fragrance oil having internal designation 0/253597 but representative of common fragrance oils) were hand mixed at room temperature. 78.0% water was added while stirring gently at room temperature for about 25 minutes to make 132.92 g of product. The viscosity of the mixture was initially as low as that of water, but after about 15 to 20 minutes it increased gradually. After about 30 to 40 minutes the product took on an emulsion-like consistency. The product was white in appearance, had a pH of 6.8, and was very sensitive to shear.

An absorbent pad was placed on the bottom of a receptacle of similar size and shape to popular commercial gel type vapor generators. The emulsion was poured into the receptacle and the receptacle was then placed in a conventional air freshening unit, a battery powered ventilation system with intermittent fan.

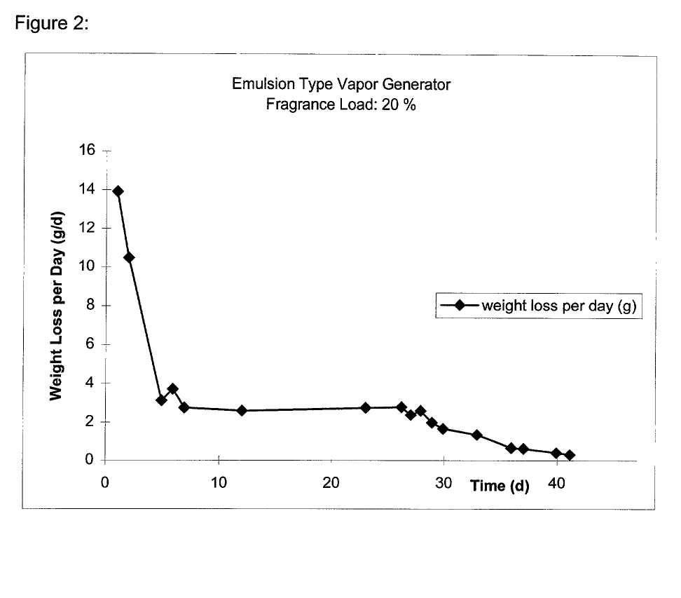

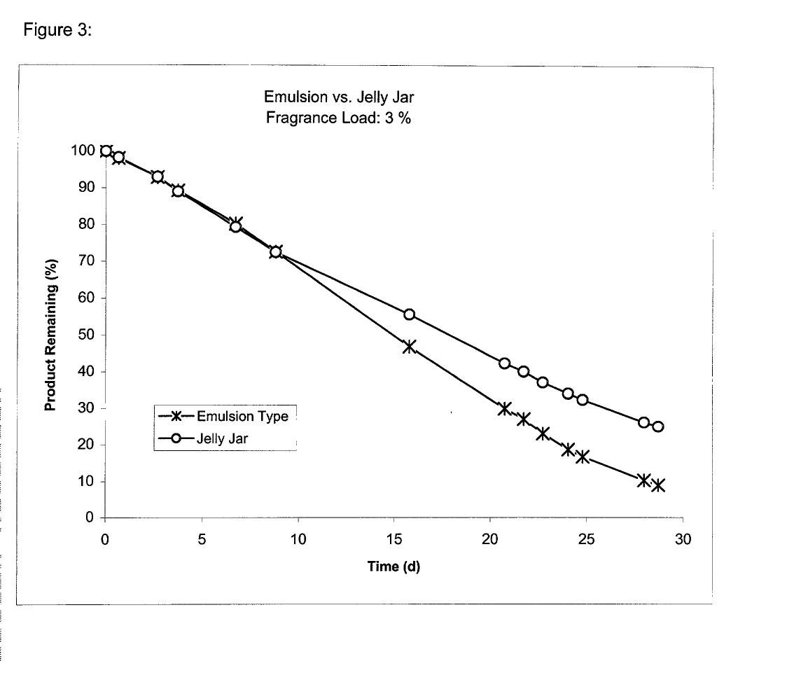

The emulsion product gradually decreased in size along its vertical axis. During the entire 30 day period the fragrance composition was found to have good fragrance intensity and good fragrance balance of top notes, middle, and bottom notes. The results in terms of weight loss per day, weight without tare, and total weight are set forth in FIG. 2 labeled “Emulsion Chart 1”. It can be seen that between day 5 and day 30 the weight loss per day (which corresponds to release of volatiles per day) was remarkably consistent. The loss of water contributes very much to the initial weight loss during the first five days until equilibrium is reached (as discussed above). Even on day 30 the fragrance was still evaluated as olfactively satisfactory both in terms of balance and intensity.

### Comparative Example 1

A wick type vapor generator formulated for a 56 day life cycle was compared against the emulsion type vapor generator of Example 1, which was formulated for a 34 day life cycle and which contained a proportional amount of fragrance oils. The emulsion type vapor generator provided a more intense and balanced fragrance at the end of its life cycle (day 30) than the wick type vapor generator at day 16 (first third of its life cycle).

### Comparative Example 2

An emulsion type vapor generator was formulated to contain 3.0% fragrance oils, 2.0% Salcare SC91, with water and preservative as the balance. For comparison, a jelly jar vapor generator was formulated to contain 3.0% of the same fragrance oils. The emulsion type vapor generator was found to be highly superior to the jelly jar vapor generator in terms of fragrance intensity and balance throughout the length of the study (30 day period). This was particularly true toward the end of the life cycle of both products (Table 3).

TABLE 3
Olfactive Evaluation:

| | Emulsion | |
|---|---|---|
| Time (d) | Type | Jelly Jar |
| 1 | 2.0 | 2.5 |
| 7 | 2.0 | 3.0 |
| 16 | 2.0 | 3.5 |
| 25 | 2.5 | 4.5 |
| 29 | 3.0 | 5.0 |

Key:
1: very good intensity and balance
5: very poor intensity and balance

The emulsion type vapor generator composition of the present invention was described herein with great detail with respect to an air freshener composition. However, it will be readily apparent that the invention is capable of use in a number of other applications requiring a different composition, such as an insect repellant, anti-histamine, etc. vapor generating composition. This invention has been described in its preferred form with a certain particularity inherent to air fresheners. Yet, it is understood that the present disclosure of the preferred form has been made only by way of example and that numerous changes in the details of structures and the composition of the combination may be resorted to without departing from the spirit and scope of the invention.

Now that the invention has been described,

What is claimed is:

1. An emulsion type vapor generator composition comprising:
(A) water as main ingredient in terms of weight;
(B) a hydrophobic air treatment ingredient to be vaporized, components (A) and (B) forming an emulsion; and
(C) an emulsifying agent, said emulsifying agent present in an ambient sufficient to stabilize said emulsion against coalescence yet insufficient to solidify said composition.

2. An emulsion type vapor generator composition as in claim 1, wherein said emulsifying agent is a polymer formed from monomers including at least one monomer selected from ethenoid monomers containing acrylic or methacrylic groups.

3. An emulsion type vapor generator composition as in claim 2, wherein said ethenoid monomers containing acrylic or methacrylic groups are selected from ionogenic methacrylate esters containing vinyl and tertiary amine grouping.

4. An emulsion type vapor generator composition as in claim 2, wherein said ethenoid monomers containing acrylic or methacrylic groups are selected from the group consisting of dimethylaminoethyl methacrylate (DMAEMA), dimethylaminopropyl acrylamide, diethyl-aminoethyl methacrylate and dimethylaminoethyl acrylate.

5. An emulsion type vapor generator composition as in claim 2, wherein said ethenoid monomer containing acrylic or methacrylic groups is dimethylaminoethyl methacrylate (DMAEMA).

6. An emulsion type vapor generator composition as in claim 1, wherein said hydrophobic air treatment ingredient to be vaporized is selected from the group consisting of fragrances, medicaments, decongestants, inhalants, repellants, insecticides, bactericides, and deodorants.

7. An emulsion type vapor generator composition as in claim 1, wherein said composition comprises 1.0-30.0 wt % fragrance oil; 0.5-20.0 wt % thickening agent; and water to make 100.0 wt %.

8. An emulsion type vapor generator composition as in claim 1, wherein said composition comprises 3.0-25.0 wt % fragrance oil; 1.5-10.0 wt % emulsifying agent; 0.0-25.0 wt % optional ingredients, and water to make 100.0 wt %.

9. An emulsion type vapor generator comprising:
a receptacle having a base, sides along it's length, and an opening, and an emulsion type vapor generator composition provided in said receptacle, said composition comprising (A) water as main ingredient in terms of weight, (B) a hydrophobic air treatment ingredient to be vaporized, said water and hydrophobic air treatment ingredient forming an emulsion, and components (A) and (B) forming an emulsion, and (C) a thickening agent, said thickening agent present in an amount sufficient to stabilize said emulsion against coalescence yet insufficient to solidify said composition.

10. An emulsion type vapor generator as in claim 9, wherein said receptacle has a constant cross-sectional area along at least a portion of its length.

11. An emulsion type vapor generator as in claim 9, wherein said opening is provided with a removable lid.

12. An emulsion type vapor generator as in claim 9, wherein said emulsifying agent is a polymer formed of monomers including ethenoid monomers containing acrylic or methacrylic groups.

13. An emulsion type vapor generator as in claim 12, wherein said ethenoid monomers containing acrylic or methacrylic groups are selected from the group consisting of dimethylaminoethyl methacrylate (DMAEMA), dimethylaminopropyl acrylamide, diethyl-aminoethyl methacrylate and dimethylaminoethyl acrylate.

14. An emulsion type vapor generator as in claim 9, further including a pad of absorbent material provided inside said receptacle.

15. An emulsion type vapor generator as in claim 9, wherein said hydrophobic air treatment ingredient to be vaporized is selected from the group consisting of fragrances, medicaments, decongestants, inhalants, repellants, insecticides, bactericides, and deodorants.

16. A method of treating air, said method comprising:
(1) formulating an emulsion type vapor generator composition comprising (A) water as main ingredient in terms of weight, (B) a hydrophobic air treatment ingredient to be vaporized, said water and hydrophobic air treatment ingredient forming an emulsion, and components (A) and (B) forming an emulsion, and (C) an emulsifying agent, said emulsifying agent present in an amount sufficient to stabilize said emulsion against coalescence yet insufficient to solidify said composition;
(2) providing said emulsion type vapor generator composition in a receptacle having a base, sides, and an opening;
(3) exposing the upper surface of said emulsion type vapor generator composition to the atmosphere to thereby cause vaporization of water and active agent and generally one-dimensional shrinkage of said emulsion volume along its vertical axis.

17. An emulsion type vapor generator composition as in claim 17, wherein said emulsifying agent is a polymer formed from monomers including at least one monomer selected from ethenoid monomers containing acrylic or methacrylic groups.

18. An emulsion type vapor generator composition as in claim 17, wherein said ethenoid monomers containing acrylic or methacrylic groups are selected from ionogenic methacrylate esters containing vinyl and tertiary amine grouping.

19. An emulsion type vapor generator composition as in claim 17, wherein said ethenoid monomers containing acrylic or methacrylic groups are selected from the group consisting of dimethylaminoethyl methacrylate (DMAEMA), dimethylaminopropyl acrylamide, diethyl-aminoethyl methacrylate and dimethylaminoethyl acrylate.

20. A refill packet for a vapor generator, said packet comprising a flexible enclosure means containing an emulsion type vapor generator composition comprising:
(A) water as main ingredient in terms of weight;
(B) a hydrophobic air treatment ingredient to be vaporized, components (A) and (B) forming an emulsion; and
(C) an emulsifying agent, said emulsifying agent present in an amount sufficient to stabilize said emulsion against coalescence yet insufficient to solidify said composition.
